(19) United States
(12) Patent Application Publication
(10) Pub. No.: US 2025/0082660 A1
(43) Pub. Date: Mar. 13, 2025

(54) Active Ingredient Group For Treating Constipation

(71) Applicant: Centre for Chinese Herbal Medicine Drug Development Limited, Hong Kong (CN)

(72) Inventors: Zhaoxiang Bian, Hong Kong (CN); Lin Zhu, Hong Kong (CN); Jingchun Shi, Hong Kong (CN); Ka Wing Cheng, Hong Kong (CN); Yan Yan Lam, Hong Kong (CN); Ziwan Ning, Hong Kong (CN); Chengyuan Lin, Hong Kong (CN)

(21) Appl. No.: 18/530,302

(22) Filed: Dec. 6, 2023

(30) Foreign Application Priority Data
Sep. 11, 2023 (CN) 202311163687.6

(51) Int. Cl. A61K 31/7048; A61K 31/05; A61K 31/192; A61K 31/352; A61K 36/185; A61K 36/575; A61K 36/65; A61K 36/708; A61K 36/752; A61P 1/10

(52) U.S. Cl. CPC A61K 31/7048; A61K 31/05; A61K 31/192; A61K 31/352; A61K 36/185; A61K 36/575; A61K 36/65; A61K 36/708; A61K 36/752; A61P 1/10

(57) ABSTRACT

The present invention belongs to the field of medical technology and specifically relates to an active ingredient group for treating constipation. An active ingredient group for treating constipation, characterized in that the active ingredient group includes rhein, hesperetin, albiflorin, and magnolol. The key target combination of constipation includes RXRA, CYP1A1, CYP1A2 and PLA2G4. The active ingredient group for treating constipation provided in this application can effectively prevent and treat constipation by regulating bile acid secretion, steroid hormone biosynthesis, glycerophospholipid metabolism, and linoleic acid metabolism by establishing strong combination between the active ingredient group and the key target combination of constipation. This application also discloses the biomarkers for the diagnosis and treatment of constipation.

### CROSS-REFERENCE TO RELATED APPLICATIONS

This non-provisional application claims priority under 35 U.S.C. § 119 (a) on patent application No. 202311163687.6 filed in China on Sep. 11, 2023, the entire contents of which are hereby incorporated by reference.

### TECHNICAL FIELD

The present invention belongs to the field of medical technology, especially involving an active ingredient group for treating constipation.

### BACKGROUND OF THE INVENTION

Constipation is a highly heterogeneous disorder, involving myriad pathophysiological factors. Traditional Chinese Medicine (TCM) is equally complex in its multiple-component, multiple-target, and multiple-mechanism properties. Furthermore, TCM has been used for thousands of years for this common human complaint. Thus, TCM is a promising alternative treatment for constipation in the modern era as well. The Chinese herbal formula, MaZiRenWan (MZRW), also called hemp seed pills, was first recorded in the TCM classic book, “Discussion of Cold-induced Disorders” (Shang-HanLun). It has been used for constipation for more than 2,000 years. MZRW comprises six herbs: Fructus Cannabis (Huo Ma Ren, HMR), Radix et Rhizoma Rhei (Da Huang, DH), Radix Paeoniae Alba (Bai Shao, BS), Semen Armeniacae Amarum (Ku Xing Ren, KXR), Fructus Aurantii Immaturus (Zhi Shi, ZS), and Cortex Magnoliae Officinalis (Hou Pu, HP). Randomized controlled trial (RCT) studies showed that it is effective and has no side effects on various subtypes of constipation. An RCT of 291 patients with functional constipation showed that, compared with Senna (Senna is an FDA-approved, nonprescription, single-ingredient, first-line laxative on the market), it significantly increased colonic transit; it reduced the number and severity of global constipation symptoms, including straining; it improved evacuation; and it showed better sustainable effect.

CDD-2101 is a standardized MZRW formulation, manufactured with quality control according to the Good Manufacturing Practice (GMP) standards. We used CDD-2101 to investigate the mechanism(s) of MZRW. However, the underlying mechanism(s) of CDD-2101 in treating constipation have not been fully elucidated. We still need further research on the targets and active ingredients of CDD-2101, to improve the accuracy and effectiveness of constipation diagnosis and treatment, and we hope to provide the basis for developing CDD-2101 into a constipation treatment that is more effective with fewer side effects than the drugs currently available.

### SUMMARY OF THE INVENTION

In view of this, this application provides an active ingredient group for treating constipation to solve the technical problems defined in the background technical section of this application.

The active ingredient group for treating constipation provided in this application to address its technical issues are:

An active ingredient group for treating constipation, characterized by:

the active ingredient group includes rhein, hesperetin, albiflorin, and magnolol.

Preferably, the active ingredient group can act on the key target combination of constipation, which includes RXRA, CYP1A1, CYP1A2 and PLA2G4.

Preferably, there is a strong binding between the active ingredient group and the key target combination of constipation.

Preferably, the active ingredient group can regulate bile acid secretion, steroid hormone biosynthesis, glycerophospholipid metabolism, and linoleic acid metabolism.

Preferably, the biomarkers for treating constipation using the active ingredient group are 8,11,14-eicosatrienoic acid, cortisol, Lysophatidylethanolamine, and chenodeoxycholic acid. Preferably, the active ingredient group comes from CDD-2101.

Preferably, the CDD-2101 comprises:
Fructus Cannabis (Huo Ma Ren, HMR),
Radix et Rhizoma Rhei (Da Huang, DH),
Semen Armeniacae Amarum (Ku Xing Ren, KXR),
Cortex Magnoliae Officinalis (Hou Pu, HP),
Radix Paeoniae Alba (Bai Shao, BS),
Fructus Aurantii Immaturus (Zhi Shi, ZS).
   Benefits of this Application:

The active ingredient group for treating constipation provided in this application can effectively prevent and treat constipation by regulating bile acid secretion, steroid hormone biosynthesis, glycerophospholipid metabolism, and linoleic acid metabolism by establishing strong combination between the active ingredient group and the key target combination of constipation. At the same time, the biomarkers for the diagnosis and treatment of constipation have also been disclosed.

The following is a detailed introduction to the technical solution and effects of this application, combined with the accompanying drawings and specific implementation methods of the specification.

### BRIEF DESCRIPTION OF DRAWINGS

FIG. 1, Number of fecal pellets (A) and their water content (B) during the 0-9 hours after treatment in four groups of mice: control, model (loperamide-induced), CDD-2101 (loperamide induced+CDD-2101 treatment), Senna (positive control, loperamide induced+Senna treatment). All data are expressed as mean±SEM (Standard Error of the Mean), n=6, *p<0.05, ** p<0.01.

FIG. 2, PLS-DA score plots of the metabolic profiles in fecal samples from the four groups of mice in positive (A) and negative (B) mass spectrometry scan mode.

FIG. 3, Volcano plots of metabolites from Model vs Control (A) and CDD-2101 treatment vs Model (B); (C) heatmaps of differential metabolites identified from control vs. model vs. CDD-2101; (D) dumbbell charts corresponding to the heat maps, control vs. model (blue) and CDD-2101 vs. model (red); (E) targeted analysis of BAs in feces (n=6); (F) metabolic pathways enriched by differential metabolites in fecal samples.

FIG. 4, Network pharmacology analysis. Chromatograms of CDD-2101 group (A) and Senna group (B); fecal samples after the corresponding treatments (C and D). (E) PPI network map of potential target genes. (F) Hub genes identified from the PPI network. (G) Top 24 remarkably enriched results from GO analysis for biological function of potential target genes. (H) Top 20 remarkably enriched KEGG analysis for the signaling pathways and metabolic pathways of potential target genes.

FIG. 5, Comprehensive network analysis. (A) Compound (metabolite)-reaction-enzyme-gene networks for key metabolites and targets (hexagon for compound, and circle for gene, yellow nodes for key compounds and gens). (B) Compound (ingredient)-target-pathway-metabolite network (different classes of nodes with different colors and shapes).

FIG. 6, The detailed molecular docking result of the strongest binding affinity pair. The IC50 data were reported in in vitro enzyme assays by previous studies.

FIG. 7, Integrated pathway by which rhein, hesperetin, albiflorin and magnolol affect four major components of the physiology of constipation (BA and steroid biosynthesis, glycerophospholipid and linoleic acid metabolism). The first and the second arrow near metabolites from left to right denote changes in control vs. model and model vs. CDD-2101 groups. AA (arachidonic acid), LA (linoleic acid).

FIG. 8, RSD % distribution in samples metabolomics profiling of QC samples.

FIG. 9, The validation of PLS-DA of metabolomics.

FIG. 10, (A) The heatmaps of differential metabolites identified from control vs. model vs. Senna; (B) the dumbbell charts correspond to the left heat maps, control vs. model (blue) and Senna vs. model (red).

### DETAILED DESCRIPTION OF THE INVENTION

The specific validation experiments and theoretical studies using CDD-2101 as a constipation treatment drug will provide a detailed explanation of the active ingredient group for treating constipation disclosed in this application.

### 1. Materials and Methods

### 1.1 Reagents and Materials

CDD-2101, manufactured by PuraPharm (Nanning) Pharmaceutical Co., Ltd, is a water extract from six botanical raw materials including Huomaren (35.7%; w/w), Dahuang (17.9%; w/w), Kuxingren (17.9%; w/w), Houpo (10.7%; w/w), Baishao (8.9%; w/w) and Zhishi (8.9%; w/w). The manufacturing process and quality control of CDD-2101 were not changed from that of marketed MZRW, which was approved and supervised by National Medical Products Administration of China. Senna tablet laxatives were purchased from Mannings store in Hong Kong with the labeled content of sennosides as 8.6 mg in each tablet. Loperamide hydrochloride was purchased from Chengdu Must Bio-technology Co., Ltd. Methanol (Honeywell, France), acetonitrile (DUKSAN, Korea), and formic acid (VWR, USA) were of HPLC grade. The reference standards of synephrine, amygdalin, albiflorin, paeoniflorin, naringin, hesperidin, aloe emodin, rhein, emodin, aloe emodin, honokiol, magnolol, chrysophanol and physcion were purchased from China National Institutes for Food and Drug Control. Cannabisin B, hesperetin, linoleic acid, benzaldehyde, benzoic acid, naringenin, sennoside A, and sennoside B were purchased from ChemFace (China). The purity of all standards was ≥98.0% (HPLC).

### 1.2 Animals and the Loperamide-Induced Acute Model

Six-week-old male (18-22 g) C57BL/6J Mus musculus were obtained from Laboratory Animal Services Center, The Chinese University of Hong Kong. All mice were housed in an environmentally controlled room (22±1° C.; ˜ 50% relative humidity) with a 12-h light-dark cycle, and fed a normal rodent diet and water. Animal care was approved by the Committee on the Use of Human and Animal Subjects in Teaching and Research, Hong Kong Baptist University (REC/19-20/0300).

Mice were randomly assigned to the following four groups (n=6 per group): control group, loperamide group, loperamide+CDD-2101 group, and loperamide+Senna group. The mice in the control group were only orally treated with Milli-Q water. The other groups were orally administrated with loperamide (2.5 mg/kg body weight). CDD-2101 and Senna were administered based on the clinical dosages of MZRW and Senna, respectively, used in constipation patients. The CDD-2101 group was orally administered with 30 g/d/kg body weight. The Senna group was given 8 tablets/day.

### 1.3 Fecal Sample Collection and Preparation

All fecal pellets produced during the first 5 min were discarded. Then fecal samples were collected, and the frequency was recorded for the next 9 h. Fecal pellets were put into tubes set in dry ice. After collection, they were stored at −80° C. for further use. Gastrointestinal motility was calculated from pellet frequency. The secretion was determined by pellet water content.

Fecal samples were weighed, placed in a 1.5 mL Eppendorf tube, lyophilized and homogenized. Approximately 15 mg of powdery fecal were added to 500 μL of pre-cooled 80% methanol solution. Then they were homogenized with beads for 5 min and incubated at −80° C. for 4 hours. The extract was centrifuged (18,000×g, 4° C.) for 10 min. The supernatant was collected and dried. 100 μL pre-cooled methane/water (1:1) was added to the supernatant, and the mixture vortexed for 5 min, then centrifuged (18,000×g, 4° C.) for 10 min. The supernatant was collected and stored at −80° C. until analysis.

### 1.4 Metabolomics Analysis

Metabolomics analysis was performed on a 1290 Infinity ultra-high performance liquid chromatographic (UHPLC) column coupled with 6546 Q-TOF mass spectrometer (UHPLC/Q-TOF-MS) (Agilent Technologies, Santa Clara, CA, USA). Chromatographic separation was performed on a ZORBAX Eclipse Plus C18 column (2.1 mm×100 mm, 1.8 μm, Agilent) at 40° C. The mobile phase consisted of 0.1% of formic acid in water as the mobile phase A, 0.1% of formic acid in acetonitrile as the mobile phase B. The gradient program was as follows: 1% B (0-1.0 min), 1 to 100% B (1.0-12.5 min), 100-100% B (12.5-14.5 min), 100-1% B (14.5-14.7 min) and maintained at 1% B (17.7 min). The injection volume was 8 μL; the flow rate was 0.4 mL/min. MS analysis was carried out in both positive and negative ion modes. The electrospray ionization (ESI) source was set with the following parameters: gas temperature, 300° C.; drying gas, 8 L/min, nebulizer, 45 psi; sheath gas temperature, 350° C., sheath gas flow, 8 L/min; capillary voltage, 3.0 kV. MS1 full scan range was m/z 80-1000. MS2 full scan parameters were as follows: full scan range, m/z 40 to 1000; collision energy, 10, 20 and 40 eV, respectively.

### 1.5 Metabolite Identification and Metabolic Pathway Analysis

The acquired MS1 raw data was converted to mzXML format using MSConvert GUI software from the ProteoWizard toolset. The intensities were corrected for signal drift and batch effect by fitting a locally quadratic (loess) regression model to the median intensity of pooled QC samples. For multivariate statistical analysis, supervised partial least squares discrimination analysis (PLS-DA) was performed using SIMCA-P software. The features with fold change (FC)>1.2 or <0.8, and p-value <0.05 were considered to be potentially differential compounds. Identification of these compounds was firstly performed by comparing the accurate MS and MS/MS (ppm <5) with an online database [Human Metabolome Database (HMDB)]. Some were verified by authentic standards. Heat maps were displayed using the pheatmap package in R. Metabolic pathway analysis was performed by MetaboAnalyst 5.0 (https://www.metaboanalyst.ca/) based on the Kyoto Encyclopedia of Genes and Genomes (KEGG, https://www.genome.jp/kegg/).

### 1.6 Network Pharmacology Construction

First, the phytochemical compounds in CDD-2101 and feces were characterized using the UHPLC/Q-TOF-MS with the same parameters as for the metabolomics analysis described in section 1.4. Potential active compounds were selected because of their frequent use in pharmacology study, and their high abundance in CDD-2101 and feces. Those compounds were confirmed by commercial standards. Second, the biological targets of these compounds were gathered by searching in BindingDB (https://www.bindingdb.org/) and STITCH (http://stitch.embl.de/). The candidate targets of constipation were gathered by searching on the keyword “constipation” in GeneCards (https://www.genecards.org/). Third, these targets were manually checked in full texts of relevant references. The confirmed proteins were considered to be the predicted targets of CDD-2101 against constipation.

These proteins were imported into UniProtKB (http://www.uniprot.org/) to standardize the names of proteins and related genes. A Protein-Protein Interaction (PPI) network was built via the STRING database (https://string-db.org/). Hub genes were obtained using CytoHubba in Cytoscape 3.9.1. The herb-compound-target network was established and visualized in Cytoscape. The pathway and Gene Ontology (GO) and KEGG enrichment analysis of potential targets were analyzed by R packages “org.Hs.eg.db”, “clusterProfiler”, “Cairo”, and “ggplot2”, and further analysis was set as p-value <0.05.

### 1.7 Integrated Network Analysis of Metabolomics and Network Pharmacology

To find the altered key metabolites, related pathways, and their targets, various networks were constructed and analyzed. First, the differential metabolites identified by metabolomics and the genes predicted by network pharmacology were imported into the Cytoscape equipped with MetScape; this yielded the compound (metabolites)-reaction-enzyme-gene network. This construction was performed to visualize the interactions and relationships between metabolites, enzymes and genes. Second, the medicine, herbs, active compounds (ingredients), genes, pathways, and metabolites were imported into the Cytoscape to obtain the network. Third, the key metabolites, genes and active compounds were obtained by overlapping the two integrated networks.

### 1.8 Molecular Docking

Docking of the key active compounds and genes was explored using CB-Dock online molecular docking (http://clab.labshare.cn/cb-dock/php/blinddock.php). PDP files for receptors were downloaded from https://www.rcsb.org/, and the active ingredient SDF files were downloaded from the PubChem database (http://pubchem.ncbi.nlm.nih.gov). All the prepared files were uploaded to the CB-Dock website. After determining the docking pocket coordinates, molecular docking and conformational scoring were performed using CB-dock. The lower the score is, the more stable is the ligand binding to the receptor, which was used for preliminary evaluation of the binding activity of the compound to the target.

### 1.9 Statistical Analysis

Statistical analysis was performed by one-way ANOVA followed by Dunnett's multiple comparisons using GraphPad Prism 7 (GraphPad Software, Inc., La Jolla, CA, USA). A p-value of <0.05 was considered statistically significant.

### 2. Results

### 2.1 CDD-2101 Treatment Ameliorates Constipation

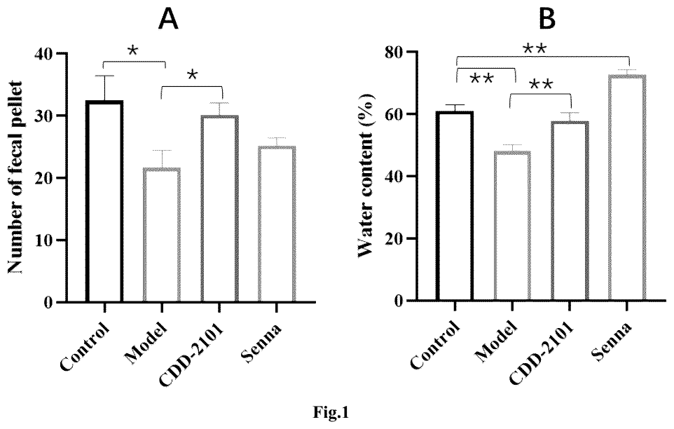

Constipation and the therapeutic effects of CDD-2101 and of positive control Senna were assessed by the number and water content of fecal pellets. As shown in FIG. 1A, the cumulative number of fecal pellet significantly decreased in loperamide group compared with the control group (p<0.05, 21.5±2.7 vs 32.5±3.9 pellets/9 h). However, the fecal pellet numbers significantly increased in CDD-2101-treated group compared with the model group (p<0.05, 30.2±1.9 pellets/9 h) and was increased to the same degree as the control group. The fecal pellet numbers slightly increased in the Senna group (positive control) compared with the model group.

As shown in FIG. 1B, the fecal pellet water content (%) was significantly decreased in the loperamide group when compared with the control group (p<0.005, 48.2±2.1 vs 61.1±2.0/9 h). In contrast, the water content was significantly increased in the CDD-2101 group compared with the model group (p=0.017, 57.9±2.7/9 h) and was improved to the same extent as the control group. In addition, the water content was significantly higher in the Senna group compared with other groups (p<0.01, 72.7±1.8/9 h), including the control group. The animals (16.7%) in Senna group showed slight diarrhea. The above results demonstrated that intake of both CDD-2101 and Senna can relieve constipation, while Senna showed an adverse effect, namely diarrhea.

### 2.2 Metabolomics Profiling Analysis

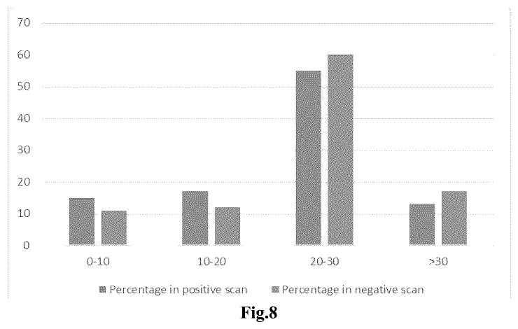

More than 10,000 features were extracted from all the fecal pellets. The stability and repeatability of metabolomics profiling analysis were evaluated by the performance of QC samples. As shown in FIGS. 8, 87.1% and 82.9% of metabolites had an RSD %<30% in the positive and negative scan modes, respectively. These data indicate that the current method is stable and repeatable.

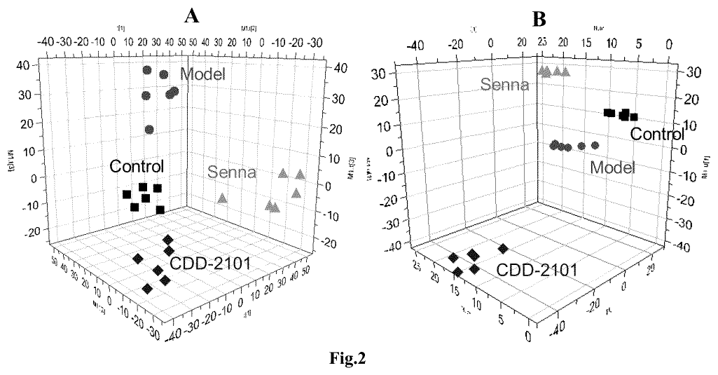

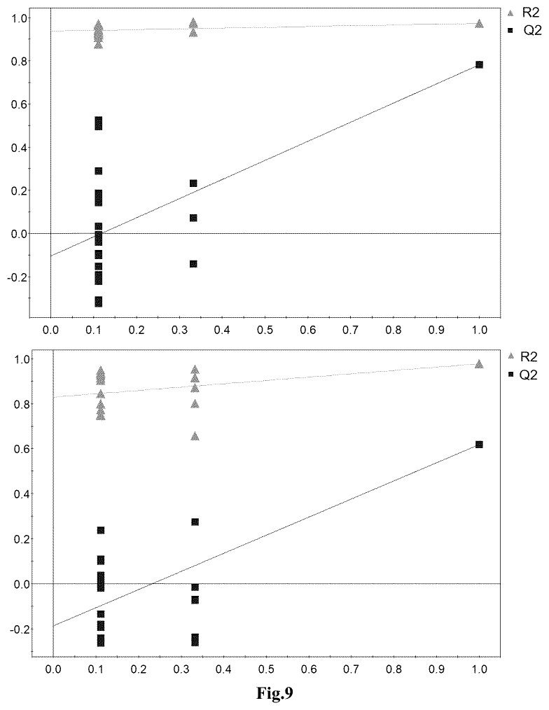

To investigate the overall metabolic changes among the four groups as reflected by the feces samples, we performed PLS-DA multivariate statistical analysis. As shown in FIG. 2, PLS-DA score plots show that the samples from different groups clustered away from each other, indicating different metabolic conditions. The parameters of R2X, R2Y and Q2 in PLS-DA of fecal samples (positive scan mode) were 0.469, 0.994 and 0.867, respectively. The parameters of R2X, R2Y and Q2 in PLS-DA of fecal samples (negative scan mode) were 0.466, 0.993 and 0.825, respectively (FIG. 9). The high levels of R2Y and Q2 showed good explanative ability of sample classification information and predictive capability of the PLS-DA models. These results indicated that loperamide and CDD-2101 caused clear metabolic variations.

### 2.3 Differential Metabolite Identification and Pathway Analysis

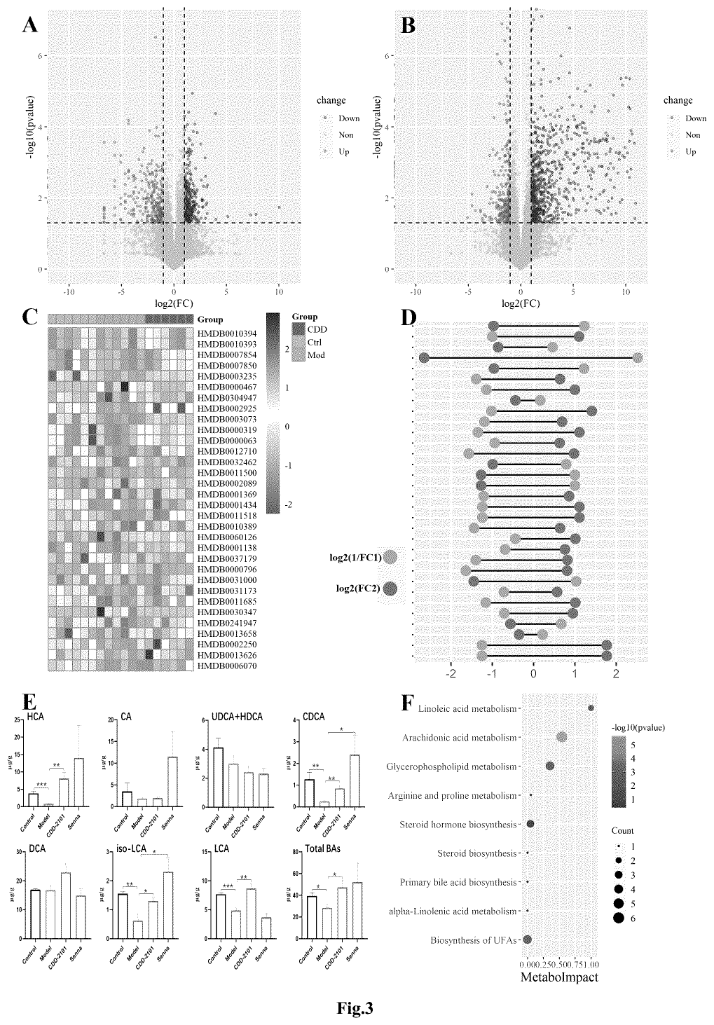

Based on FC and p value cutoff, 1305 and 1835 features were differentially expressed in the fecal samples between the Model vs Control and CDD-2101 treatment vs Model, respectively. After loperamide induced (Model vs Control), 508 features were downregulated, while 797 features were upregulated. After CDD-2101 treatment, 1057 features were upregulated (CDD-2101 vs Model), while 778 features were downregulated. The results were visualized in volcano plots (FIG. 3A-B).

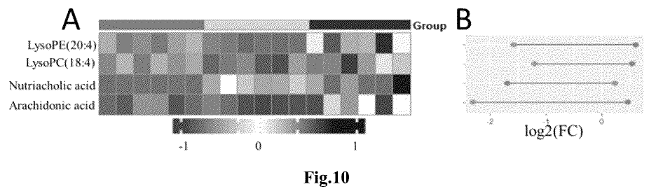

According to HMDB, 52 metabolites were identified and differentially expressed in feces between control group and model group. The levels of 33 metabolites were successfully restored by CDD-2101 treatment (Table 1). To visualize the variation of metabolites, we plotted heat maps and dumbbell charts. FIGS. 3C and 3D show that the candidate metabolites were changed in the loperamide model group, and most of them were reversed in the CDD-2101 group, indicating that the CDD-2101 treatment had alleviated metabolic perturbation. In contrast, fewer metabolites were rescued related to the Senna group, and the metabolic perturbations were not regulated to the same extent as the control group (FIG. 10).

It is worth mentioning that relatively more bile-related metabolites were significantly altered in the CDD-2101 and Senna groups. Therefore, we carried out targeted quantification analysis of bile acids (BAS) (FIG. 3E). Among the tested BAs, the total BAs, hyocholic acid (HCA), chenodeoxycholic acid (CDCA), iso-isolithocholic acid (iso-LCA) and lithocholic acid (LCA) were decreased in the feces from the model mice group when compared with the control group. Interestingly, the levels in the group treated by CDD-2101 were improved to comparable levels of control group. Although BA-related metabolism was improved by Senna treatment, some metabolite levels (CDCA, iso-LCA and HCA) were even significantly higher than in the control group. These results suggest that the CDD-2101 may protect against constipation by regulating the BA-related metabolism pathway.

To further explore the constipation-related metabolic pathways regulated by CDD-2101, we imported these differential metabolites to MetaboAnalyst 5.0. As shown in FIG. 3F, 12 metabolic pathways were enriched in the feces, including arachidonic acid metabolism, glycerophospholipid metabolism, biosynthesis of unsaturated fatty acids, linoleic acid metabolism, arginine and proline metabolism, primary BA biosynthesis, and steroid hormone biosynthesis, etc.

TABLE 1
SPECIFCATION
Details of differential metabolites in fecal samples (FC1: control vs. model, FC2: CDD-2101 vs. model).

| No. | RT(min) | Precursor MS | MS/MS | Metabolites | HMDBID | FC1 | FC2 |
|---|---|---|---|---|---|---|---|
| 1 | 1.014 | 188.0558 | 55.0187/67.0187 | N-Acetylglutamicacid | HMDB0001138 | 2.65 | 2.07 |
| 2 | 1.575 | 350.109 | 87.0089/128.0350 | N-Acetyl-4-O-acetylneuraminicacid | HMDB0000796 | 0.36 | 0.29 |
| 3 | 1.640 | 130.05 | 41.0391/42.0344/68.050 | Pyrrolinehydroxycarboxylicacid | HMDB0001369 | 1.82 | 1.99 |
| 4 | 2.880 | 288.1193 | 110.0718 | N-Ribosylhistidine | HMDB0002089 | 0.51 | 0.62 |
| 5 | 3.156 | 212.0915 | 94.0652/124.0757 | 3-Methoxytyrosine | HMDB0001434 | 2.61 | 3.39 |
| 6 | 4.098 | 191.0532 | 129.0552 | 3-Dehydroquinicacid | HMDB0012710 | 1.67 | 1.87 |
| 7 | 4.698 | 233.1536 | 41.0387/133.1016 | (Z)-8-Decene-4,6-diyn-1-y13-methylbutanoate | HMDB003 1000 | 1.83 | 2.00 |
| 8 | 4.954 | 199.1334 | 43.0189/59.0138 | 3-Nonanon-1-ylacetate | HMDB0037179 | 0.76 | 0.77 |
| 9 | 5.106 | 426.2596 | 44.0496 | LysoPE(14:0/0:0) | HMDB0011500 | 0.42 | 0.50 |
| 10 | 5.296 | 167.107 | 77.0381/91.0543 | Penty12-furylketone | HMDB0032462 | 0.61 | 0.41 |
| 11 | 5.319 | 191.0816 | 146.0606 | 6-Hydroxy-1H-indole-3-acetamide | HMDB0031173 | 1.64 | 2.96 |
| 12 | 6.217 | 363.2166 | 273.186 | 18-Hydroxycorticosterone | HMDB0000319 | 1.74 | 1.48 |
| 13 | 6.217 | 363.2166 | 273.186 | Cortisol | HMDB0000063 | 1.74 | 1.48 |
| 14 | 6.339 | 297.1119 | 55.0542/93.0701/ | DHAP(8:0) | HMDB0011685 | 2.09 | 3.03 |
| 15 | 6.774 | 268.1705 | 91.0543/130.0656 | N-(p-Hydroxyphenethyl)actinidine | HMDB0030347 | 0.01 | 0.21 |
| 16 | 7.137 | 351.216 | 351.2166 | ProstaglandinG2 | HMDB0003235 | 4.80 | 1.98 |
| 17 | 7.171 | 429.3006 | 55.0541 | 25-HydroxyvitaminD3-26,23-lactone | HMDB0060126 | 0.22 | 0.45 |
| 18 | 7.512 | 391.285 | 337.2522/145.1009 | Nutriacholicacid | HMDB0000467 | 0.04 | 0.14 |
| 19 | 8.218 | 416.3158 | 81.0700/95.0860 | N-Stearoylmethionine | HMDB0241947 | 2.00 | 1.87 |
| 20 | 9.688 | 391.2843 | 337.2522/145.1009 | Chenodeoxycholicacid | HMDB0000518 | 5.18 | 3.42 |
| 21 | 9.978 | 502.2932 | 44.0495 | LysoPE(20:4(8Z,11Z,14Z,17Z)/0:0) | HMDB0011518 | 1.55 | 2.02 |
| 22 | 10.571 | 372.2906 | 107.0855/215.1805 | Docosahexaenoylethanolamide | HMDB0013658 | 1.97 | 1.64 |
| 23 | 10.866 | 516.306 | 86.0961/184.0724 | LysoPC(18:4(6Z,9Z,12Z,15Z)/0:0) | HMDB0010389 | 1.78 | 2.23 |
| 24 | 11.534 | 379.282 | 41.0386 | Nordeoxycholicacid | HMDB0304947 | 0.57 | 0.49 |
| 25 | 11.560 | 279.2326 | 261.2218/233.2269 | Gamma-linolenicacid | HMDB0003073 | 2.72 | 2.49 |
| 26 | 11.568 | 344.2799 | 55.0542/95.0855 | Dodecanoylcarnitine | HMDB0002250 | 1.53 | 2.35 |
| 27 | 11.815 | 439.2823 | 81.0699/109.1014 | LysoPA(18:0/0:0) | HMDB0007854 | 1.75 | 1.87 |
| 28 | 11.815 | 439.2823 | 81.0699/109.1014 | LysoPA(0:0/18:0) | HMDB0007850 | 1.75 | 1.87 |
| 29 | 11.842 | 307.263 | 289.2526/259.2426 | 8,11,14-Eicosatrienoicacid | HMDB0002925 | 1.64 | 2.34 |
| 30 | 11.893 | 546.3524 | 86.0964 | LysoPC(20:3(8Z,11Z,14Z)) | HMDB0010394 | 0.50 | 0.58 |
| 31 | 11.893 | 546.3524 | 86.0964 | LysoPC(20:3(5Z,8Z,11Z)) | HMDB0010393 | 0.50 | 0.58 |
| 32 | 13.181 | 376.3193 | 57.0699 | Adrenoylethanolamide | HMDB0013626 | 1.79 | 2.72 |
| 33 | 13.248 | 337.2722 | 107.0855/109.1012 | Pregnanetriol | HMDB0006070 | 1.62 | 2.93 |

PA: phosphatidic acid,
PC: phosphocholine,
PE: phatidylethanolamine.

### 2.4 Network Pharmacology

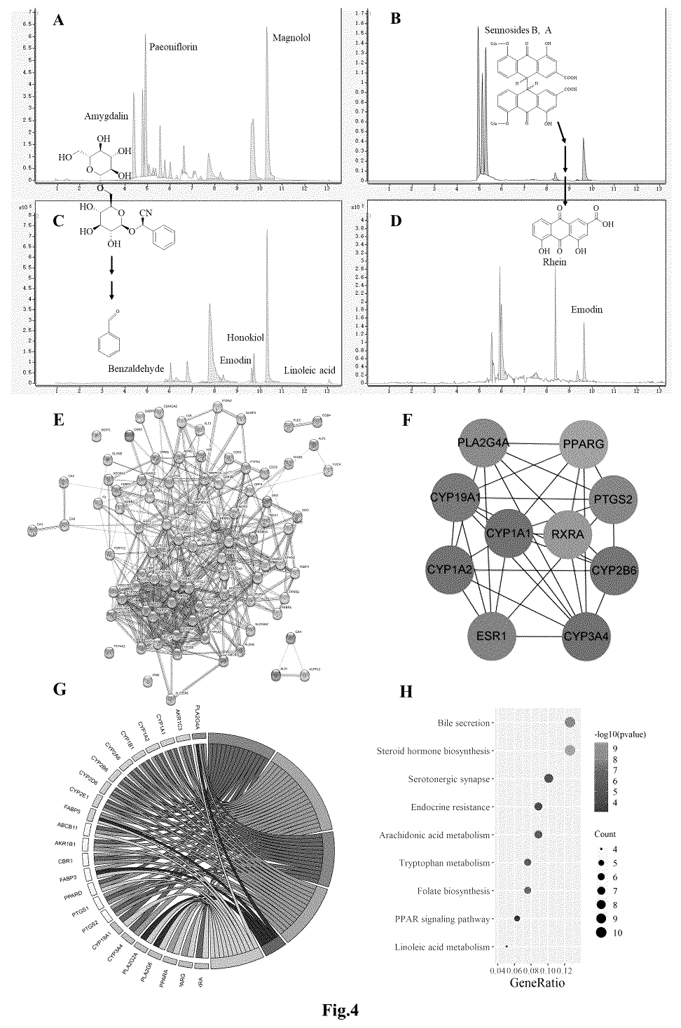

A combination strategy of network pharmacology and fecal phytochemistry provided novel insights into the potential targets and pathways of active ingredients derived from CDD-2101 against constipation. Based on the phytochemical analysis in this study, 17 prototype compounds were identified from CDD-2101. It is reported that they are the corresponding main ingredients in the six herbs of CDD-2101. In many studies, these compounds have been frequently detected in blood and feces, and are widely regarded as compounds with pharmacological activities. We also identified four metabolites derived from these prototype ingredients in feces. As a result, a total of 21 compounds (prototype+metabolite) were selected as candidates for network pharmacology study (FIGS. 4A and C, Table 2). We found that some of the prototype ingredients—namely paeoniflorin, albiflorin, amygdalin, hesperidin, and naringin with relatively lower bioavailability could be metabolized to benzoic acid, benzaldehyde, hesperetin and naringenin to exert biological activities, which is consistent with previous studies. The sennosides A and B (major ingredients of Senna) could be metabolized to rhein, which are also a major ingredient of CDD-2101 as well (FIG. 4A-D, Table 2).

TABLE 2
The major ingredients of CDD-2101 and Senna.
The selected ingredients from CDD-2101 Senna as
candidates for network pharmacology analysis.

| RT (min) | Name | m/z | Formula | Herb |
|---|---|---|---|---|
| 1.424 | Synephrine | 168.1019 | C₉H₁₃NO₂ | ZS |
| 4.398 | Amygdalin | 456.1523 | C₂₀H₂₇NO₁₁ | KXR |
| 4.779 | Albiflorin | 525.1614 | C₂₃H₂₈O₁₁ | BS |
| 4.913 | Paeoniflorin | 525.1614 | C₂₃H₂₈O₁₁ | BS |
| 4.924 | Sennoside B | 861.1884 | C₄₂H₃₈O₂₀ | Senna |
| 5.26 | Sennoside A | 861.1884 | C₄₂H₃₈O₂₀ | Senna |
| 5.368 | benzoic acid | 121.0295 | C₇H₆O₂ | BS |
| 5.45 | Naringin | 579.1719 | C₂₇H₃₂O₁₄ | ZS |
| 5.563 | Hesperidin | 609.1825 | C₂₈H₃₄O₁₅ | ZS |
| 5.652 | Neohesperidin | 609.1825 | C₂₈H₃₄O₁₅ | ZS |
| 5.794 | Benzaldehyde | 105.0346 | C₇H₆O | KXR |
| 5.95 | Cannabisin A | 593.1929 | C₃₄H₃₀N₂O₈ | HMR |
| 6.029 | Cannabisin B | 595.2086 | C₃₄H₃₂N₂O₈ | HMR |
| 7.142 | Naringenin | 271.0617 | C₁₅H₁₂O₅ | ZS |
| 7.368 | Hesperitin | 301.0718 | C₁₆H₁₄O₆ | ZS |
| 8.239 | Aloe emodin | 269.0455 | C₁₅H₁₀O₅ | DH |
| 8.339 | Rhein | 283.0248 | C₁₅H₈O₆ | DH, Senna |
| 9.616 | Emodin | 269.0455 | C₁₅H₁₀O₅ | DH, Senna |
| 9.728 | Honokiol | 265.1234 | C₁₈H₁₈O₂ | HP |
| 10.288 | Magnolol | 265.1234 | C₁₈H₁₈O₂ | HP |
| 10.904 | Chrysophanol | 253.0506 | C₁₅H₁₀O₄ | DH |
| 11.329 | Physcion | 283.0612 | C₁₆H₁₂O₅ | DH |
| 13.083 | Linoleic acid | 279.2328 | C₁₈H₃₂O₂ | HMR, HXR |

The italics word indicates the metabolite of the prototype ingredient(s) of the corresponding herb HMR: Fructus Cannabis (Huo Ma Ren), DH: Radix et Rhizoma Rhei (Da Huang), BS: Radix Paeoniae Alba (Bai Shao), KXR: Semen Armeniacae Amarum (Ku Xing Ren), ZS: Fructus Aurantii Immaturus (Zhi Shi), HP: Cortex Magnoliae Officinalis (Hou Pu)

Subsequently, we gathered 122 targets of the candidate active ingredients of CDD-2101 from the BindingDB and STITCH, and we gathered 5272 targets related to constipation from the GeneCards database. After matching analysis, 86 compounds of CDD-2101 were identified as potential targets against constipation. Further, to select the hub genes of CDD-2101 against constipation, we constructed PPI network by Cytoscape. FIG. 4E gives a view of all the relationships within 83 targets (the other three genes were disconnected). We calculated relationship degrees using CytoHubba. Combining the scores of 10 computational methods, the top 10 genes were considered as hub genes (RXRA, CYP1A1, PLA2G4, PTGS2, ACHE, ESRI, CYP1A2, CYP19A1, CYP2B6, PPARG) of CDD-2101 against constipation (FIG. 4F, Table 3).

TABLE 3
The evaluation of hub genes by CytoHubba

| Name | MCC | MNC | Degree | EPC | Bottleneck | EcCentricity | Closeness | Radiality | Betweenness | Stress | Rank |
|---|---|---|---|---|---|---|---|---|---|---|---|
| CYP1A1 | 245922 | 26 | 52 | 14.588 | 1 | 0.31325 | 50.16667 | 4.05195 | 146.5545 | 12128 | 1 |
| CYP1A2 | 250006 | 30 | 60 | 14.849 | 2 | 0.31325 | 52.5 | 4.12518 | 366.7513 | 19240 | 2 |
| ESR1 | 49089 | 31 | 64 | 14.081 | 13 | 0.31325 | 53.5 | 4.14959 | 846.8168 | 37688 | 3 |
| PTGS2 | 2126 | 28 | 56 | 14.403 | 5 | 0.31325 | 52.16667 | 4.14959 | 530.8391 | 26488 | 4 |
| PLA2G4A | 1040 | 12 | 24 | 9.49 | 1 | 0.31325 | 42.83333 | 3.85667 | 41.69315 | 3552 | 5 |
| ESR2 | 1036 | 13 | 26 | 9.616 | 6 | 0.23494 | 42.08333 | 3.77124 | 84.92436 | 5168 | 6 |
| PPARG | 807 | 29 | 60 | 13.061 | 11 | 0.31325 | 53.16667 | 4.17399 | 784.1018 | 29512 | 7 |
| PTGS1 | 220 | 10 | 20 | 7.642 | 1 | 0.23494 | 40.91667 | 3.75904 | 31.85226 | 2456 | 8 |
| RXRA | 60 | 11 | 22 | 7.468 | 2 | 0.23494 | 40.5 | 3.69801 | 56.59551 | 2776 | 9 |
| ACHE | 9 | 4 | 10 | 4.471 | 1 | 0.31325 | 36 | 3.52715 | 10.39204 | 576 | 10 |

To determine the constipation relief and intestinal protective functions of the potential targets, we performed GO and KEGG pathway enrichment analyses. The top biological functional components in GO analysis were fatty acids, lipids, cholesterol, steroids, and hormones related to biological processes (FIG. 4G). According to the KEGG enrichment analysis (FIG. 4H), the significantly affected pathways were focusing on steroid hormone biosynthesis, arachidonic acid metabolism, retinol metabolism, bile secretion, folate biosynthesis, linoleic acid metabolism, tryptophan metabolism, the PPAR signaling pathway, endocrine resistance, IL-17 signaling pathway, and glycerophospholipid metabolism. Obviously, the pathways predicted by network pharmacology included most of the pathways enriched by the metabolomics. The high consistency of pathway analysis results enabled us to build a comprehensive network analysis platform to decipher key targets and mechanisms against constipation by CDD-2101.

### 2.5 Integrated Analysis of Metabolomics and Network Pharmacology

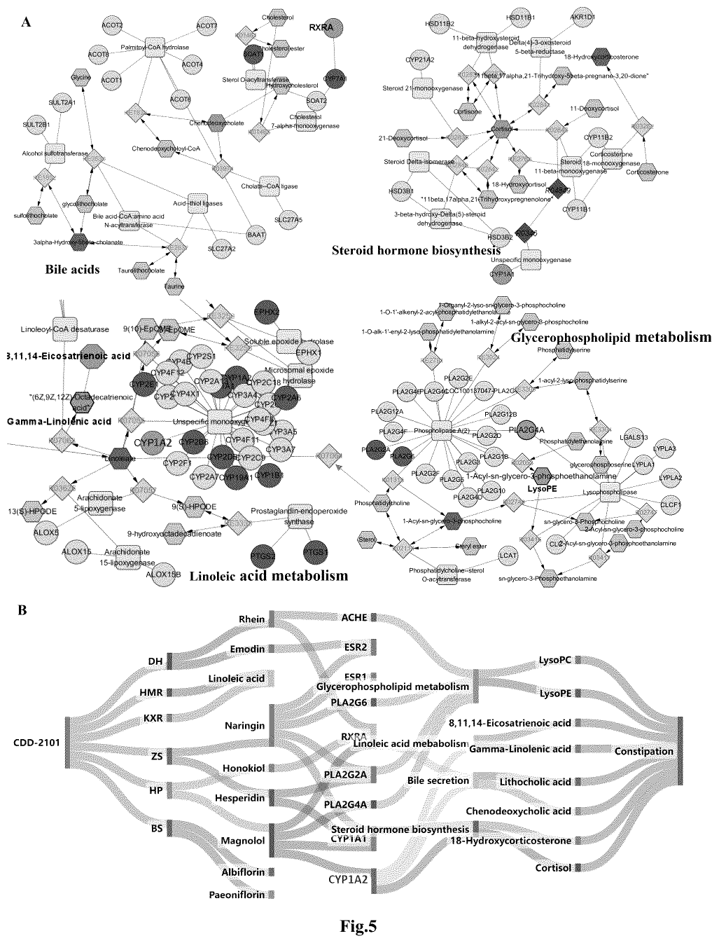

To obtain a comprehensive view of the mechanisms by which CDD-2101 treats constipation, we constructed two integrated interaction networks based on metabolomics analysis and network pharmacology results, and matched them by Cytoscape. First, based on the differential metabolites and the potential targets, the compound-reaction-enzyme-gene network showed that RXRA, CYP1A1, CYP1A2, and PLA2G4 might be the key targets, and that 8,11,14-eicosatrienoic acid, 18-hydroxycorticosterone, cortisol, LysoPE (20: 4 (8Z,11Z,14Z,17Z)/0:0) (LysoPE), and CDCA might be the key metabolites regulated by CDD-2101 (FIG. 5A). Second, based on the compound-target-pathway-metabolite network, we found that bile secretion, steroid hormone biosynthesis, glycerophospholipid metabolism, and linoleic acid metabolism might be the key pathways, with albiflorin, rhein, hesperetin and magnolol being the key active ingredients (FIG. 5B). Finally, by matching the two comprehensive networks, we found the metabolites, targets, pathways and ingredients that may play essential roles in the therapeutic effect of CDD-2101 on constipation. They are summarized in Table 4. Further validation is needed.

TABLE 4
Key active ingredients, targets, metabolites
and pathways for CDD-2101.

| Pathway | Metabolite | Target | Active ingredient |
|---|---|---|---|
| Glycerophospholipid | LysoPE | PLAG4A | Albiflorin |
| metabolism | | | |
| Steroid hormone | Cortisol | CYP1A1 | Hesperetin |
| biosynthesis | | | |
| Bile secretion | CDCA | RXRA | Rhein |
| Linoleic acid | 8,11,14-Eico- | CYP1A2 | Magnolol |
| metabolism | satrienoicacid | | |

Hesperetin: metabolite type of Hesperidin in CDD-2101.
Rhein: metabolite type in Senna, while prototype in CDD-2101.

### 2.6 Molecular Docking Validation

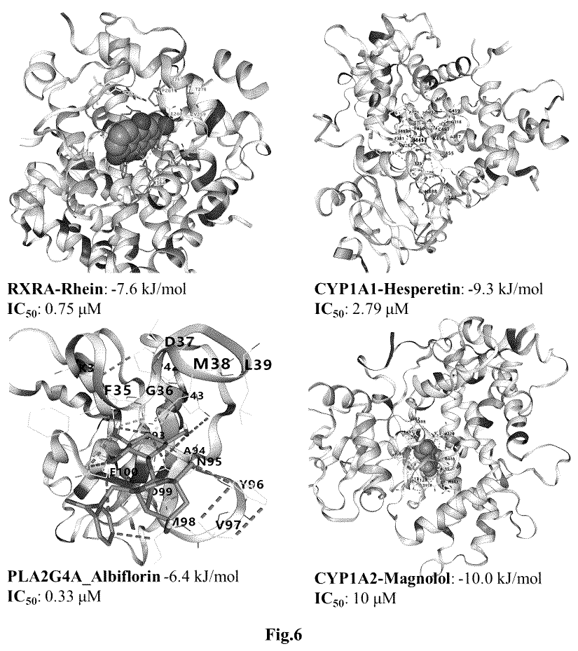

To further investigate the possibility of interaction among the key active compounds and the targets, we performed molecular docking studies (FIG. 6). Generally, binding affinity lower than-5 KJ/mol is regarded as indicating good interaction. The docking analysis of RXRA showed that rhein made hydrogen-bonding interactions with E260, ILE318, VAL259 and R371 at the active site, and the binding energy was-7.6 KJ/mol. In the interaction with CYPIAI, hesperetin made hydrogen-bonding interaction with GLY459, PHE381, VAL382, THR385, ILE386, and the binding energy was-9.3 KJ/mol. In the interaction with PLA2G4, albiflorin formed hydrogen bonds with ASP93, ALA94, ASN95, TYR96 and the energy was-6.4 KJ/mol. In the interaction with CYP1A2, magnolol formed hydrogen bonds with THR224, ALA370, ARG372, GLU374, and the energy was −8.1 KJ/mol. These docking results indicate high affinities between the active compounds and the targets. Actually, in vitro enzyme assays have shown their strong binding affinities.

### 3.Discussion

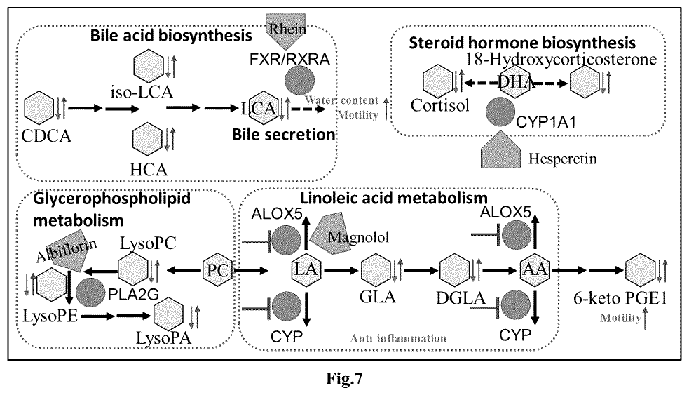

Constipation is as common as it is hard to treat, much less cure. Research has shown that myriad pathophysiological factors are involved in its etiology. Certain TCM formulas have successfully treated constipation, perhaps because it involves multiple components targeting multiple aspects. Until now, science has not been able to isolate, identify, and understand the multi-component/multi-targeting aspects of Chinese herbs Metabolomics and network pharmacology are two powerful new tool that can be used for this purpose. Many studies using fecal metabolomics have found that both constipated human phenotypes and animals exhibit differences in the metabolic profiles and metabolic biomarker levels compared with control groups. Here, we identified 33 significant differential metabolites in feces, as well as their related pathways. Network pharmacology greatly improves the screening of metabolites of how CDD-2101 treats constipation and explains the biochemical mechanisms involved. By our novel integrated network analytical approach, combining metabolomics with network pharmacology (FIG. 7), we found that the four major active ingredients (rhein, hesperetin, albiflorin, and magnolol) from the herbs strongly bind to four key targets (RXRA, CYP1A1, CYP1A2, and PLA2G4, respectively), regulating related metabolic pathways (Bile acid secretion, steroid hormone biosynthesis, glycerophospholipid metabolism, and linoleic acid metabolism, respectively) to alleviate constipation, and that the key metabolites (8, 11, 14-eicosatrienoic acid, cortisol, LysoPE, and CDCA) can serve as biomarkers for diagnosis and treatment. This strategy provides a feasible solution to verify the results of the two approaches. It is also practicable to screen metabolites and targets in other natural medicine development.

Previous study has confirmed that constipation or diarrhea can be caused by the abnormal delivery of BAs to the colon. The BAs have important functions in regulating the water content of feces. For example, some BAs such as deoxycholic acid increase the secretion of water and electrolytes in colon. Other BAs can influence colon motor activity. For example, decreased levels of secondary BAs reduce the release of 5-hydroxytryptamine from enterochromaffin cells, thereby affecting gut motility to induce constipation. Here, our metabolomics results showed that both the secondary BAs, such as LCA, and total BAs were significantly lower in the model group compared with the control group. In contrast, total BAs were significantly increased in the CDD-2101 treatment group. The network pharmacology showed that some active ingredients in CDD-2101 may mainly act on target RXRA to regulate BA metabolism. These compounds include rhein, naringenin, synephrine, and albiflorin. The major active ingredients in Senna (sennosides) have been demonstrated to be metabolized to rhein in the colon. Therefore, both CDD-2101 and Senna may mainly act on RXRA to regulate BA metabolism to treat constipation. In the liver, FXR/RXR downregulates CYP7A1 expression, resulting in decreased BA synthesis and secretion. Molecular docking shows that rhein has the strongest affinity for RXRA among these active ingredients. Previous studies have demonstrated that rhein has good binding activity with FXR/RXR to increase the BA synthesis and bile secretion. The metabolites related to primary BA synthesis, secondary BA synthesis and bile secretion were restored to normality after CDD-2101 treatment. However, some excess BAs may induce diarrhea, which may explain the slight diarrhea in the Senna group, in which the levels of CDCA and iso-LCA were significantly higher than in the control group.

Reduced levels of steroid hormones are associated with severe constipation. In the model group, downstream metabolites of the steroid hormone biosynthesis pathway, cortisol and 18-hydroxycorticosterone, were down-regulated while the CDD-2101 successfully restored normal levels. Steroid hormones are derived from cholesterol. In the biosynthesis of progesterone, cholesterol is firstly converted to pregnenolone by a bifunctional enzyme complex. Progesterone and estrogen are implicated in the development of constipation. Hesperetin, magnolol and naringenin have been reported to bind to the CYP1A1 enzyme to regulate steroid hormone biosynthesis to alleviate the constipation. Naringin and hesperidin, the major compound in CDD-2101, has been demonstrated to be metabolized to naringenin and hesperetin, respectively, in the colon. Therefore, we can conclude that the prototype compounds and their metabolite type can be used to regulate the steroid hormone levels to treat constipation.

The metabolites identified in CDD-2101 group are classified as LysoPA, LysoPC and LysoPE; all three are closely related to glycerophospholipid metabolism. These metabolites, including their precursor phosphatidylcholine, are abundant in the gastrointestinal mucus barrier, where its hydrophobic properties are thought to play a role in maintaining mucus integrity. We found that albiflorin and magnolol stimulated cytosolic phospholipase A2 (cPLA2, PLA2G4) phosphorylation. This stimulation, in combination with the binding of hesperidin and aloe emodin to the ACHE enzyme, can effectively regulate this pathway to protect the intestinal mucus barrier.

Considerable evidence shows that linoleic acid metabolism is closely associated with constipation and related treatment. However, the specific process and mechanism are not clear. Here, we found that the gamma-linolenic acid and dihomo-gamma-linolenic acid (its downstream metabolite) were significantly downregulated in the model group and upregulated in the CDD-2101 group. The two metabolites facilitate generation of M2 macrophages and inhibit production and action of IL-6, IL-1β, and TNF-α, which are crucial to determining the final status of anti-inflammatory events. Furthermore, they can be introduced into the arachidonic acid metabolism and finally metabolized to 6-ketoprostaglandin E1 (6-keto PGE1), which is a stable prostacyclin (PGI2) metabolite. This metabolite, like PGI2, can promote the contraction of gastrointestinal smooth muscle to increase motility and alleviate constipation. It should be noted that LA and AA are the hub nodes of the two metabolic pathways. Interestingly, magnolol has been proved to strongly inhibit the CYP1A2 and ALOX5 enzymes in vitro and in vivo, which may explain how CDD-2101 treatment successfully restored metabolites.

We also conducted a comparison study of Senna using our comprehensive metabolomics-network pharmacology platform, analyzing the metabolites, related pathways, targets and active ingredients. We found that the major ingredients sennoside A and B were metabolized to the active ingredient rhein, which may bind to FXR/RXRA dimer to regulate BA-related metabolism pathways to alleviate constipation, just as CDD-2101 does. This is supported by the differential metabolites and the therapeutic index. However, senna group also showed some adverse effects (slight diarrhea), and the related BA metabolites were restored too higher than the control group. Previous study has confirmed that constipation or diarrhea can be caused by the abnormal delivery of BAs to the colon. Treating constipation with a single ingredient with a single target may result in over-regulated related pathways. Overall, treating constipation with multiple ingredients addressing multiple targets should mean better homeostatic control of the pathways involved.

In this present application, we first developed a novel integrated method based on metabolomics and network pharmacology to elucidate the biochemical mechanism(s) of complex natural medicines. We then used the method to study CDD-2101, a manufactured version of a traditional Chinese medicine formula. We found that the four major active ingredients from the formula's component herbs strongly bind to four key targets, which have been validated by our molecular docking and previous studies with in vitro and in vivo. Third, the bile acid secretion, steroid hormone biosynthesis, glycerophospholipid metabolism, and linoleic acid metabolism were regulated to alleviate constipation. Finally, we compared CDD-2101 with the first-line, single-ingredient laxative Senna, and found CDD-2101 was more effective with fewer side effects. Our metabolomics results suggest the multi-factorial activity of CDD-2101 explains its superior efficacy.

The above provides a detailed explanation of the technical solution and effects of the present application in conjunction with the accompanying drawings and specific embodiments in the specification. It should be noted that the specific embodiments disclosed in the specification are only the preferred embodiments of the present application, and technical personnel in the field can also develop other embodiments based on this; Any simple deformation and equivalent replacement that does not deviate from the innovative concept of this application are covered by this application and fall within the scope of protection of this patent.

1. An active ingredient group for treating constipation, characterized by:
the active ingredient group includes rhein, hesperetin, albiflorin, and magnolol.

2. The active ingredient group for treating constipation according to claim 1, characterized in that:
the active ingredient group can act on the key target combination of constipation, which includes RXRA, CYP1A1, CYP1A2 and PLA2G4.

3. The active ingredient group for treating constipation according to claim 1, characterized in that:
there is a strong binding between the active ingredient group and the key target combination of constipation.

4. The active ingredient group for treating constipation according to claim 1, characterized in that:
the active ingredient group can regulate bile acid secretion, steroid hormone biosynthesis, glycerophospholipid metabolism, and linoleic acid metabolism.

5. The active ingredient group for treating constipation according to claim 1, characterized in that:
the biomarkers for treating constipation using the active ingredient group are 8, 11, 14-eicosatrienoic acid, cortisol, lysophatidylethanolamine, and chenodeoxycholic acid.

6. The active ingredient group for treating constipation according to claim 1, characterized in that:
the active ingredient group comes from CDD-2101.

7. The active ingredient group for treating constipation according to claim 6, characterized in that:
the CDD-2101 comprises:
Fructus Cannabis,
Radix et Rhizoma Rhei,
Semen Armeniacae Amarum,
Cortex Magnoliae Officinalis,
Radix Paeoniae Alba,
Fructus Aurantii Immaturus.
